(19) United States
(12) Patent Application Publication
(10) Pub. No.: US 2007/0134783 A1
(43) Pub. Date: Jun. 14, 2007

(54) Immature unialgal culture strain

(76) Inventors: Hirotaka Kakita, Takamatsu-shi (JP); Hiroshi Kamishima, Takamatsu-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/594,899

(22) PCT Filed: Mar. 30, 2005
(86) PCT No.: PCT/JP05/06167

§ 371 Date: Sep. 29, 2006

(30) Foreign Application Priority Data
Mar. 31, 2004 (JP) 2004-108563

(51) Int. Cl. C12N 1/12

(52) U.S. Cl. 435/257.1

(57) ABSTRACT

It is intended to provide a novel unialgal culture strain showing a high culture efficiency of a large-sized red alga which is immature, can be stored and cultured over a long period of time and has at least one of the following properties, i.e., producing a physiologically active substance at a high yield, showing a high growth speed of the alga body, and being highly capable of absorbing nutritional salts. Namely, an immature unialgal culture strain originating in a large-sized red marine alga which is characterized by showing no matured female gametophyte in nature but showing matured tetrasporophyte alone and grows in a natural marine water area containing fresh water. This unialgal culture strain is constructed by collecting the matured sporophyte, cutting the sporophyte and allowing to stand to thereby release spores, culturing the released spores and continuously proliferating and culturing even after the growth of an upright body from the germinated spore.

### TECHNICAL FIELD

The present invention relates to: a novel unialgal culture strain derived from a marine macroalga of red algae which is immaturable even after long-term storage or continued culturing and is exceedingly resistant against adherence of other algae; a method for producing the unialgal culture strain; and an alga body obtained by growing the unialgal culture strain.

### BACKGROUND ART

Since terrestrial bioresources are now at a risk of being exhausted due to deforestation and so on, an important challenge for Japan, a country poor in natural resources, is to search useful marine resources and facilitate the use thereof. Particularly, marine macroalgae growing in special environments such as the ocean sometimes contain special components that are not seen in terrestrial organisms, and these special components have been utilized as raw materials for foods and industrial products [“Cultivation Studies on Marine Alga Resources (Kaiso Shigen Yoshoku-gaku in Japanese)”, Tokuda, H., Ohno, M., Ogawa, H., Midori Shobo, 1987, p. 35-66; and “Food Processing and Ingredients (Shokuhin Kaihatsu in Japanese)”, 1984, vol. 19, p. 43-48]. In recent years, several novel biologically active substances have been found from the divided fractions of marine macroalgae or from components separated therefrom. As a result, marine macroalgae have received attention as raw materials for fine chemicals (“Kaiyo Monthly”, 1995, vol. 27, p. 13-21 and “Kaiyo Monthly”, 1995, vol. 27, p. 34-39).

Components contained in marine macroalgae vary qualitatively or quantitatively according to the time in the season when the marine algae grow (“Hydrobiologia”, 1993, vol. 260/261, p. 541-547). Therefore, a culture method which precisely regulates the growing season, for example indoor culture which precisely regulates environmental factors, is required for the purpose of producing useful components. However, the low growth speed of marine algae or the large-scale consumption of filtered seawater becomes a barrier and makes the large-scale indoor culture of marine macroalgae very difficult.

Specifically, condition setting is important for the indoor culture of algae, and this condition setting requires marine alga samples for growth experiments, while it is difficult to directly use naturally growing marine macroalgae in the experiments of growth evaluation of marine macroalgae.

This is because the growth speeds of symbiotic microorganisms and so on adhering to marine macroalgae are often higher than those of the marine macroalgae under artificial culture conditions, and the resulting abnormally proliferating microorganisms and so on influence the growth of the marine macroalgae. Although drug treatment or unialgal culture strain preparation methods are known for removing such adhering symbiotic microorganisms, the latter method is more preferable than the drug treatment method because of its low damages done to alga bodies.

To develop useful components from algae, unialgal culture strains must be obtained every year because algae are generally withered after maturation. An immaturable culture strain, if any, is never matured and withered even after continued culturing for a long time, and as such, stands in no need of obtaining fresh culture strains every year. Of marine macroalgae, for example a less matured marine alga strain belonging to the genus Ulva is known as to green algae, while this kind of marine alga strain is not known so far as to red algae.

On the other hand, a method is generally known, wherein upright bodies are kept standing and stored under slow growth conditions in the preliminary step toward the growth of a unialgal culture strain, and a unialgal culture strain is grown and cultured from the upright bodies. The growth of a necessary amount of unialgal culture strain from the upright bodies usually requires much time, for example 2 to 4 weeks for marine algae belonging to the genus of Gracilaria sp., during which the experiment is inevitably interrupted.

It is also possible that growing using a unialgal culture strain alone and the growth of a unialgal culture strain from upright bodies are concurrently performed to save treatment time. However, this case has disadvantages such as complicated procedures, increased scale of culture facilities, and enormous labor required.

Thus, the emergence of a unialgal culture strain which can be grown and cultured at once when required or is immaturable and thereby continuously culturable has been demanded strongly in this technical field.

Red algae have particularly received attention by reason of their high yields of hemagglutination agents that cause mitogenic stimulation triggering the growth or proliferation of lymphocytes in the resting period, evaluate the immunogenic potentials of patients with various diseases including AIDS, or promote the division of lymphocytes in LAK therapy, a novel cancer therapy.

A method for cleaning a contaminated sea area by culturing a sterile marine alga belonging to green algae, for example sea lettuce (JP2000-254685A) and a method for culturing sea lettuce as a raw material for foods or pharmaceutical drugs on the ocean or in a solar dome (JP11-289894A and JP2004-97003A) have been proposed as methods for artificially culturing marine algae.

However, marine algae belonging to the green alga genus of Ulva sp. are flat and membranous in shape and have the following disadvantages (1) to (4):

(1) they can not be cultured in plural layers due to their membranous shapes; (2) the alga bodies are weak and easily torn as compared with a cylindrical red alga Gracilaria verrucosa; (3) they can not be cultured in the state immobilized on a carrier due to their easily torn alga bodies. Moreover, the collection thereof is not easy, and the torn algae cause contamination; and (4) the sea lettuce when exceeding a size of 30 cm squares is difficult to bend or disperse by stirring and sustains damages by receiving the sunlight, causing reduction in the growth speed. Therefore, the growth speed can not be recovered unless the sea lettuce must be collected and cut (JP2000-254685A and JP2004-97003A).

In general, the growth of green algae requires stronger light intensity than that required by marine algae of red algae. To utilize products associated with the growth of marine algae or the function of the growing marine algae, facilities or conditions that keep stronger light intensity are generally needed in the use of marine algae of green algae than in the use of marine algae of red algae.

Sterile sea lettuce is rotten and disappears as a result of decomposition, and next year, a remaining portion thereof grows with increases in nutritional salt concentration and results in unusual proliferation. This cycle is probably repeated every year. Indeed, sterile sea lettuce accumulating in the seashore has been responsible for environmental pollution. Since sea lettuce containing water becomes relatively easily rotten, for example becomes rotten after 1 day, it is pointed out that the sea lettuce needs dehydration and drying immediately after the collection thereof from media or seawater (JP2000-254685A and JP2004-97003A).

By contrast, red algae, for example Gracilaria verrucosa, can be immobilized on carriers and cultured in large amounts by virtue of their robust, less cleavable alga bodies, and they are suitable for large-scale indoor culture because of being easily controlled and collected, resisting damages by the light received even when getting larger in the dimensions of the algae, growing even under weak light and resisting decay, causing no environmental pollution, and capable of being cultured in layers of the alga bodies which are filamentous in shape.

### DISCLOSURE OF THE INVENTION

Under such circumstances, an object of the present invention is to provide a novel unialgal culture strain showing a high culture efficiency of a macroalga of red algae which is immaturable and storable and culturable over a long period of time and has at least one of the following properties: properties of producing a biologically active substance in a high yield, showing a high growth speed of the alga body, and being capable of readily absorbing nutritional salts.

The present inventors have conducted various studies on a unialgal culture strain from a marine macroalga of red algae and have consequently found that a unialgal culture strain derived from a marine macroalga of red algae growing in a natural seawater area with intermixing of fresh water and having characteristics that no female gametophytes are detectable as matured bodies in nature and only tetrasporophytes are detectable as matured bodies, is immaturable over a long period of time and is exceedingly resistant against adherence of other algae even after long-term continued culturing. The present inventors have completed the present invention on the basis of these findings.

Specifically, the present invention provides: an immaturable unialgal culture strain derived from a marine macroalga of red algae growing in a natural seawater area with intermixing of fresh water and having characteristics that no female gametophytes are detectable as matured bodies in nature and only tetrasporophytes are detectable as matured bodies; a method for producing an immaturable unialgal culture strain characterized by the steps of collecting matured sporophytes of a marine macroalga of red algae growing in a natural seawater area with intermixing of fresh water and having characteristics that no female gametophytes are detectable as matured bodies in nature and only tetrasporophytes are detectable as matured bodies, keeping the sporophytes in seawater as cut open to cause release of the spores and culturing the released spores to continue growing and culturing after sprouting of upright bodies from germinated spores; and an alga body obtained by growing the immaturable unialgal culture strain.

The immaturable unialgal culture strain means a unialgal culture strain which is immaturable even after 3 years or longer of continued culturing under normal culture conditions and produces a biologically active substance similar to that produced by the unialgal culture strain of the marine alga immediately after the preparation thereof. The immaturable unialgal culture strain also means a strain of the marine alga which is immaturable even after 3 years or longer of storage of the unialgal culture strain under non-growing culture conditions such as low nutrition, low temperature, and low light intensity, followed by 3 years or longer of continued culturing under normal culture conditions, and has at least one of the properties similar to those of the unialgal culture strain of the marine alga immediately after the preparation thereof, that is, properties of producing a biologically active substance in a high yield, showing a high growth speed of the alga body, and being capable of readily absorbing nutritional salts.

Next, the present invention will be described in detail.

The immaturable unialgal culture strain of the present invention can be produced by using, as a raw material, a marine macroalga of red algae growing in a natural seawater area with intermixing of fresh water, particularly a seawater area where the salt content does not exceed 1.0% by mass, for example an estuary where river water joints the ocean, and having characteristics that no female gametophytes are detectable as matured bodies in nature and only tetrasporophytes are detectable as matured bodies.

In the present invention, the marine macroalga of red algae refers to a large-sized marine alga belonging to the class Rhodophyceae in the system of plant classification and has characteristics that it has chlorophyll a and phycobilin as main pigments contained therein and generates and stores floridoside and floridean starch through photosynthesis. The genera Gelidium, Gracilaria, Gigartina, Chondrus, Porphyra, and the like are included in this class. The marine macroalga of red algae used in the present invention is preferably Gracilaria verrucosa, Gracilaria chorda, or a subspecies thereof.

In the present invention, the red alga genus of Gracilaria sp. includes (1) marine algae classified into the marine alga genus of Gracilaria sp., (2) marine algae classified into Gracilariopsis sp., and (3) marine algae classified into Gracilariopsis sp. in the past.

For example, the red alga genus of Gracilaria sp. among marine algae of Japanese origin includes marine algae classified into the family Gracilariaceae of the order Gracilariales in “New Japanese Seaweed Magazine, Overview of Seaweeds of Japanese Origin (Shin Nihon Kaiso-shi Nihonsan Kaisorui Soran in Japanese), Yoshida, T., Uchida, R., pub., 1998”. These red algae also reside in cold sea but largely in warm sea. They are distributed over almost all coastal regions in Japan and used as an expander for agar, garnishing served with raw fish (sashimi), or the like.

The immaturable unialgal culture strain is obtained from the marine macroalga of red algae by the following procedures: matured portions of matured sporophytes of the marine macroalga of red algae growing in a natural seawater area with intermixing of fresh water and having characteristics that no female gametophytes are detectable as matured bodies in nature and only tetrasporophytes are detectable as matured bodies, are chopped to a length of 2 to 5 cm, preferably 3 to 4 cm, then washed with sterilized water or seawater, and kept standing in sterilized seawater for 6 to 15 hours to cause the release of the spores.

Next, the released spores are collected and separated, then inoculated into a container with a culture solution, and statically cultured at a temperature of 10 to 30° C. under light exposure and in the dark alternating at 10-to 15-hour intervals. In this context, the culture solution used is, for example sterilized seawater supplemented with usual enrichment agents for seawater.

In this way, thick upright bodies of deep color are selected after the static culture for 15 to 25 days from among upright bodies of the marine alga sprouting from germinated spores. The selected upright bodies are kept statically cultured for additional 50 to 80 days and thereby grow to a length of 10 mm.

The upright bodies are taken from the bottom of the culture container with tweezers, then inoculated to a flask, and cultured under stock culture conditions to cause the growth of alga bodies. As a result, unialgal culture strain beyond a fixed amount can be obtained.

Examples of conditions of this culture include a temperature of 15 to 30° C., light intensity of 50 to 120 μmol/m²/sec, and a light cycle including a light phase for 8 hours or longer in 24 hours. During this culture, shaking (on the order of 50 to 200 rpm) or aeration may be performed, if necessary. The culture solution may be natural seawater or artificial seawater. In some cases, the culture solution may be supplemented with marine alga growth-promoting components such as Provasoli's enrichment agents for seawater [“Research Technique for Algae (Sourui Kenkyuho in Japanese)”, Nishizawa, K., Chihara, M., ed., Kyoritsu Shuppan, Tokyo (1979), pp. 281-305].

In the present invention, the unialgal culture strain means an alga body obtained by growing upright bodies by the growing and culturing thereof.

The growth speed of the alga body can be suppressed by placing the upright body or unialgal culture strain under non-growing culture conditions such as low nutrition, low temperature, and low light intensity. It can thereby be stored and cultured at a low growth level. Therefore, such culture conditions are convenient to use when the use of the upright body or unialgal culture strain is not scheduled or when the amount of the alga body growing is desired to be controlled.

The non-growing culture conditions such as low nutrition, low temperature, and low light intensity are accomplished by, for example (1) nutritional salt concentration conditions where the total concentration of nitrate nitrogen and ammonia nitrogen and a phosphate ion concentration are 3 μM or lower and 1 μM or lower, respectively, (2) low temperature conditions where a temperature is 5 to 14° C., (3) low light intensity conditions where light intensity is 20 to 40 μmol/m²/sec, and (4) combinations of (1) to (3).

The immaturable unialgal culture strain of the present invention is immaturable even after 3 years or longer of continued culturing under culture conditions and is resistant against growing of adhering algae. In general, marine algae are withered, if worst, with increases in the number of adhering algae because the adhering algae growing faster than the marine algae ingest nutrients in media and inhibit the growth of the marine algae. However, the immaturable unialgal culture strain of the present invention is resistant against adherence of adhering algae and is therefore storable over a long period of 3 years or longer. Moreover, the immaturable unialgal culture strain of the present invention is culturable at a high growth speed in a medium, and after storage, its growth can be re-opened quickly at desired periods.

### BEST MODE FOR CARRYING OUT THE INVENTION

Next, the best mode for carrying out the present invention will be described by way of Examples. However, the present invention is not limited to these Examples by any means.

### EXAMPLE 1

Screening of red algae belonging to the genus of Gracilaria sp.

As an example of red algae belonging to the genus of Gracilaria sp., Gracilaria chorda belonging to the red alga genus of Gracilaria sp. was monthly examined for the amount (growth) of the marine alga appearing and maturation thereof at 3 locations over 3 years from April 1998 to March 2001.

The inside of the Katsuura River in the estuary of the Katsuura River, Tokushima city, Tokushima prefecture, Japan, was selected as survey location A. Hereinafter, the marine alga growing in the survey location A is referred to as a marine alga of the genus of Gracilaria sp. growing in the Katsuura River (Gracilaria chorda growing in the Katsuura River). In this site, the whole or partial community of the marine alga (Gracilaria chorda ) of the genus of Gracilaria sp. growing in the Katsuura River appeared in the tidelands at low tide of the spring.

The coast (coast adjacent to the estuary of the Yoshino River, class A river specified by the Japanese River Law) of Kawauchi-cho, Tokushima city, Tokushima prefecture, Japan, was selected as survey location B. Hereinafter, the marine alga (its adaptability to the estuary was lower than that of the marine alga of the genus of Gracilaria sp. growing in the Katsuura River) growing in this survey location B is referred to as a marine alga of the genus of Gracilaria sp. growing offshore of Kawauchi-cho, Tokushima city, Tokushima prefecture [or Gracilaria chorda (growing in the estuary of the Yoshino River)].

The Inland Sea offshore of Wadajima, Komatsushima city, Tokushima prefecture, Japan, was selected as survey location C. Hereinafter, the marine alga growing in this survey location C is referred to as Gracilaria chorda growing offshore of Komatsushima.

A change in the wet mass of the alga Gracilaria chorda per unit volume in the community of Gracilaria chorda growing in the flat surf zone (for the survey location A, the estuary that dries up at low tide) of the intertidal zone, and the number of matured individuals in all the individuals of Gracilaria chorda were examined in each survey location. In this examination, a square frame of 20 cm in length and width was placed each time at 4 locations in the community of Gracilaria chorda to determine the average value of the numbers of the matured individuals within the 4 square frames.

The maturation or immaturation of the alga body of the collected Gracilaria chorda was determined by observation with a stereoscopic microscope, based on whether or not tetrasporangium or cystocarp was formed in the alga body. The alga body where the formation of tetrasporangium was detected by the observation was identified as a matured tetrasporophyte, whereas the alga body where the formation of cystocarp was detected by the observation was identified as a matured female gametophyte. From this observation result, the number of individuals of matured tetrasporophytes with respect to the number of all the individuals of Gracilaria chorda was determined as the ratio (%) of matured tetrasporophytes. Alternatively, the number of individuals of matured female gametophytes with respect to the number of all the individuals of Gracilaria chorda was determined as the ratio (%) of matured female gametophytes. A red alga belonging to the genus of Gracilaria sp. having characteristics that no female gametophytes are detectable as matured bodies in nature and only tetrasporophytes are detectable as matured bodies, can be screened by comparing the respective results of the survey locations.

The survey result of the matured individuals of Gracilaria chorda from April 1998 to March 1999 is shown in Table 1. The survey result of the matured individuals of Gracilaria chorda from April 1999 to March 2000 is shown in Table 2. The survey result of the matured individuals of Gracilaria chorda from April 2000 to March 2001 is shown in Table 3. Numerals in each table are the average values of the values obtained in the 4 square frames of 20 cm in length and width placed in the community of Gracilaria chorda.

TABLE 1

| Year | | 1998 | | | | | | | | | 1999 | | |
|---|---|---|---|---|---|---|---|---|---|---|---|---|---|
| Month | | 4 | 5 | 6 | 7 | 8 | 9 | 10 | 11 | 12 | 1 | 2 | 3 |
| Survey | Ratio of matured | 0 | 8 | 75 | 15 | 0 | 0 | 0 | 0 | 0 | 0 | 0 | 0 |
| location A | tetrasporophytes, % | | | | | | | | | | | | |
| | Ratio of matured | 0 | 0 | 0 | 0 | 0 | 0 | 0 | 0 | 0 | 0 | 0 | 0 |
| | female gametophytes, % | | | | | | | | | | | | |
| Survey | Ratio of matured | 0 | 16 | 40 | 12 | 0 | 0 | 0 | 0 | 0 | 0 | 0 | 0 |
| location B | tetrasporophytes, % | | | | | | | | | | | | |
| | Ratio of matured | 0 | 0 | 8 | 20 | 10 | 0 | 0 | 0 | 0 | 0 | 0 | 0 |
| | female gametophytes, % | | | | | | | | | | | | |
| Survey | Ratio of matured | 0 | 10 | 48 | 16 | 0 | 0 | 0 | 0 | 0 | 0 | 0 | 0 |
| location C | tetrasporophytes, % | | | | | | | | | | | | |
| | Ratio of matured | 0 | 0 | 15 | 24 | 8 | 0 | 0 | 0 | 0 | 0 | 0 | 0 |
| | female gametophytes, % | | | | | | | | | | | | |

TABLE 2

| Year | | 1999 | | | | | | | | | 2000 | | |
|---|---|---|---|---|---|---|---|---|---|---|---|---|---|
| Month | | 4 | 5 | 6 | 7 | 8 | 9 | 10 | 11 | 12 | 1 | 2 | 3 |
| Survey | Ratio of matured | 0 | 8 | 60 | 10 | 0 | 0 | 0 | 0 | 0 | 0 | 0 | 0 |
| location A | tetrasporophytes, % | | | | | | | | | | | | |
| | Ratio of matured | 0 | 0 | 0 | 0 | 0 | 0 | 0 | 0 | 0 | 0 | 0 | 0 |
| | female gametophytes, % | | | | | | | | | | | | |
| Survey | Ratio of matured | 0 | 12 | 35 | 15 | 0 | 0 | 0 | 0 | 0 | 0 | 0 | 0 |
| location B | tetrasporophytes, % | | | | | | | | | | | | |
| | Ratio of matured | 0 | 0 | 10 | 24 | 13 | 0 | 0 | 0 | 0 | 0 | 0 | 0 |
| | female gametophytes, % | | | | | | | | | | | | |
| Survey | Ratio of matured | 0 | 8 | 44 | 12 | 0 | 0 | 0 | 0 | 0 | 0 | 0 | 0 |
| location C | tetrasporophytes, % | | | | | | | | | | | | |
| | Ratio of matured | 0 | 0 | 18 | 28 | 14 | 0 | 0 | 0 | 0 | 0 | 0 | 0 |
| | female gametophytes, % | | | | | | | | | | | | |

TABLE 3

| Year | | 2000 | | | | | | | | | 2001 | | |
|---|---|---|---|---|---|---|---|---|---|---|---|---|---|
| Month | | 4 | 5 | 6 | 7 | 8 | 9 | 10 | 11 | 12 | 1 | 2 | 3 |
| Survey | Ratio of matured | 0 | 6 | 78 | 21 | 0 | 0 | 0 | 0 | 0 | 0 | 0 | 0 |
| location A | tetrasporophytes, % | | | | | | | | | | | | |
| | Ratio of matured | 0 | 0 | 0 | 0 | 0 | 0 | 0 | 0 | 0 | 0 | 0 | 0 |
| | female gametophytes, % | | | | | | | | | | | | |
| Survey | Ratio of matured | 0 | 18 | 43 | 10 | 0 | 0 | 0 | 0 | 0 | 0 | 0 | 0 |
| location B | tetrasporophytes, % | | | | | | | | | | | | |
| | Ratio of matured | 0 | 0 | 12 | 25 | 16 | 0 | 0 | 0 | 0 | 0 | 0 | 0 |
| | female gametophytes, % | | | | | | | | | | | | |
| Survey | Ratio of matured | 0 | 6 | 48 | 10 | 0 | 0 | 0 | 0 | 0 | 0 | 0 | 0 |
| location C | tetrasporophytes, % | | | | | | | | | | | | |
| | Ratio of matured | 0 | 0 | 10 | 28 | 5 | 0 | 0 | 0 | 0 | 0 | 0 | 0 |
| | female gametophytes, % | | | | | | | | | | | | |

According to Tables 1 to 3, the “marine alga of the genus of Gracilaria sp. growing in the Katsuura River (Gracilaria chorda growing in the Katsuura River)”, which grows in the survey location A can be selected as the red alga belonging to the genus of Gracilaria sp. having characteristics that no female gametophytes are detectable as matured bodies in nature and only tetrasporophytes are detectable as matured bodies.

### EXAMPLE 2

(1) Collection and Seeding of Spores for Unialgal Culture Strain Preparation

Matured sporophytes of the marine macroalga Gracilaria chorda of the genus Gracilaria collected from the survey location A, that is, the estuary of the Katsuura River, Tokushima city, Tokushima prefecture, Japan (salt concentration: 0.5% by mass, were used as a raw material red alga belonging to the genus of Gracilaria sp. having characteristics that no female gametophytes are detectable as matured bodies in nature and only tetrasporophytes are detectable as matured bodies.

Matured portions of the matured sporophytes were chopped to a length of 30 mm, then washed with sterilized seawater, and kept standing overnight in sterilized seawater to cause release of the spores. The released spores were transferred with a sterilized Pasteur pippet to a screw tube containing 30 ml of culture solution for stock culture, and statically cultured by giving light thereto in the cycle of a 14-hour light phase and a 10-hour dark phase. The number of the spores seeded to one screw tube was set to 20. One thousand screw tubes in total were used. The static culture was performed under 11 conditions in total of (i) 6 levels of temperature (with an increment of 4° C. from 10 to 30° C.) under the constant light intensity condition of 60 μmol/m²/sec and (ii) 5 levels of light intensity (with an increment of 20 μmol/m²/sec from 20 to 100 μmol/m²/sec) under the temperature condition of 18° C.

This seawater medium was prepared by filtering seawater collected in waters with a depth of approximately 1.5 m in Yashima Bay, Takamatsu city, Kagawa prefecture, Japan, with a 0.20-μm cellulose acetate membrane filter (manufactured by Advantec Toyo), then supplementing and mixing the filtrate with 1/10 volume of distilled water, and sterilizing the mixture at 100° C. for 30 minutes, to which Provasoli's enrichment agent for seawater sterilized in advance was then added.

(2) Screening of Upright Bodies

At a moment when 21-day static culture was completed, an experimental condition that gave thick upright bodies, vibrant red pigments, and no floating matter in the culture solution was selected from among the experimental groups where germinated spores were observed. In Example 2, upright bodies germinated under the conditions of “a temperature of 18° C. and light intensity of 40 μmol/m²/sec” were selected as experimental materials.

The selected upright bodies were kept statically cultured until the resulting length of the upright bodies had reached 10 mm. In this culture, the medium was replaced with fresh one in a frequency of once every 4 weeks. In this way, upright bodies of 10 mm in length were obtained in approximately 70 days.

(3) Growing and Culturing of Upright Bodies

The upright bodies having grown into a length of approximately 10 mm were taken from the bottom of the screw tube with tweezers and inoculated into a flask to perform the growing and culturing of the upright bodies. The growing and culturing of the upright bodies were performed with aeration under conditions of a temperature of 16° C. and light intensity of 40 μmol/m²/sec (light cycle of 14-hour light phase and 10-hour dark phase) in a 1-liter round-bottomed flask containing 1 liter of culture solution. The culture solution was replaced with fresh one in a frequency of once every two weeks. The growing and culturing were performed for 70 days to cause the growth of the upright bodies. This step can be applied to the storage of upright bodies and as such, is also referred to as the step of stock culture of the upright bodies. The upright bodies that had grown in one round-bottomed flask could be divided into several 1-liter round-bottomed flasks each containing 1 liter of culture solution, to extend the period of the step of stock culture.

(4) Preliminary Culture of Unialgal Culture Strain

The upright bodies that grew in the preliminary step were cultured with aeration under conditions of a temperature of 18° C. and light intensity of 40 μmol/m²/sec (light cycle of 14-hour light phase and 10-hour dark phase) in a 1-liter round-bottomed flask containing 1 liter of culture solution. The culture solution was replaced with fresh one in a frequency of once every two weeks. In this way, the preliminary culture was performed for 35 days to obtain a unialgal culture strain.

(5) Maturation Evaluation and Growth Speed Evaluation of Unialgal Culture Strain

An alga culture test instrument capable of temperature control (temperature distribution: ±0.5° C.), light intensity control (stepless light control), and day length control was used to evaluate the maturation of the unialgal culture strain. This instrument could simultaneously be applied to fifty 500-ml Erlenmeyer flasks to culture (dimension in the tank: 1250 mm wide×720 mm deep×900 mm high). Apical fragments of 4 mm in length were prepared from the unialgal culture strain of the marine macroalga Gracilaria chorda and added at 6 fragments per Erlenmeyer flask containing 400 ml of culture seawater. Irradiation conditions were set to conditions of a 14-hour light phase and a 10-hour dark phase, and the culture solution was replaced once a week with fresh one. The number of experimental samples under the identical culture condition was 5.

Subsequently, the evaluation of maturation of the unialgal culture strain was performed with aeration under 11 conditions in total of (i) 6 levels of temperature (with an increment of 4° C. from 10 to 30° C.) under the constant light intensity condition of 60 μmol/m²/sec and (ii) 5 levels of light intensity (with an increment of 20 ,μmol/m²/sec from 20 to 100 μmol/m²/sec) under the temperature condition of 22±0.5° C.

The replacement of the culture solution and the measurement of a marine alga wet mass were performed in a clean booth. In this way, the presence or absence of maturation was determined by recording a marine alga wet mass per flask while observing the presence or absence of formation of reproductive organs such as cystocarp, tetrasporangium, or spermagonium on the marine alga surface with a microscope.

As a result, an experimental group of matured marine algae was not observed even after 12 weeks of culturing. At the point in time when the marine alga wet mass per 500-ml Erlenmeyer flask reached 0.2 g, the strain was thinned out to 0.02 g to continue the culture. However, the strain was not matured even after 3 years from the initiation of the culture (initiation of the step (5)).

Growth Rate

A relative growth rate (RGR) is expressed as R. When a marine alga wet mass at the start of culture and a marine alga wet mass after t days of culture were defined as W₀ and Wₜ, respectively, the relative growth rate is determined according to the equation R=(In Wₜ−In W₀)/t. The growth rate (%/day) was calculated by multiplying R by 100.

The growth rate of the unialgal culture strain of Gracilaria chorda (growing in the estuary of the Katsuura River) in the period of two weeks through three weeks of culture was the highest under the condition of the temperature of 22° C. and light intensity of 60 μmol/m²/sec among the experimental groups, and the value thereof was 14.4%/day.

Growth and Maturation Evaluation with 20 Liters of Culture Solution

The unialgal culture strain of Gracilaria chorda (growing in the estuary of the Katsuura River) was cultured in ten 1-liter flat-bottomed flasks and grown to a wet mass of 4 g or more. Conditions for this culture were set to the conditions that gave the highest growth rate in the culture on the scale of 400 ml of culture solution, that is, “a temperature of 22° C., light intensity of 60 μmol/m²/sec, a light cycle of a 14-hour light phase and a 10-hour dark phase, all-day aeration, and the replacement of the culture solution in a frequency of once a week”. Hereinafter, these culture conditions are referred to as the growing culture conditions.

The culture solution (seawater medium) was prepared by filtering seawater collected in waters with a depth of 1.5 m in Yashima Bay, Takamatsu city, Kagawa prefecture, Japan, with a 0.20-μm cellulose acetate membrane filter (manufactured by Advantec Toyo), then supplementing and mixing the filtrate with 1/10 volume of distilled water, and sterilizing the mixture at 100° C. for 30 minutes, to which Provasoli's enrichment agent for seawater sterilized in advance was then added. Hereinafter, this culture solution (seawater medium) is referred to as the seawater for growing culture.

The unialgal culture strain (4 g) of Gracilaria chorda (growing in the estuary of the Katsuura River) obtained by growing and culturing were inoculated into a 30-liter culture container with 20 liters of seawater for growing culture and cultured for 4 weeks under the growing culture conditions. After 4 weeks, the marine alga wet mass was increased by approximately 12 times to approximately 47 g.

No experimental group showing matured marine algae could be found even after 12 weeks of culturing. At a moment thereafter when the marine alga wet mass in the 30-liter culture container with 20 liters of seawater for growing culture had reached 300 g, the strain was thinned out to 10 g to continue the culture. Nevertheless, the unialgal culture strain was not matured even after 3 years from the initiation of the culture. The growth rates of the unialgal culture strain in 400 ml of culture solution and in 20 liters of culture solution, marine alga yields, and the presence or absence of maturation are shown in Table 4.

TABLE 4

| | Culture in 400 ml | | Culture in 20 liters | |
|---|---|---|---|---|
| | of culture solution | | of culture solution | |
| | Growth rate in the | | Change in marine | |
| | period of two weeks | Presence or | alga wet mass | Presence or |
| | through three weeks, | absence of | (in four weeks | absence of |
| | %/day | maturation | culture) | maturation |
| Unialgal culture | 14.4 | Not matured | Increase from 4 | Not matured |
| strain | | after 3 years | to 47 g in 4 | after 3 years |
| (Example 2) | | | weeks culture | |
| Unialgal culture | 8.2 | matured on the | Increase from 4 | matured on the |
| strain | | 12th week of | g to 12 g in 4 | 12th week of |
| (Comparative | | culture | weeks culture | culture |
| Example 1) | | | | |
| Unialgal culture | 7.7 | matured on the | Increase from 4 | matured on the |
| strain | | 11th week of | g to 11 g in 4 | 11th week of |
| (Comparative | | culture | weeks culture | culture |
| Example 2) | | | | |

(6) Evaluation of Activity Level of Biologically Active Substance from Unialgal Culture Strain
(a) Extraction of Water-Soluble Fraction

Gracilaria chorda (growing in the estuary of the Katsuura River) (wet mass: 25 g) obtained on the fourth week of culture was washed with 0.15 M sodium chloride aqueous solution and frozen at −30° C. A buffer solution for extraction used was 0.5 M tris(hydroxymethyl)aminomethane hydrochloride buffer solution (pH 8.2) containing 30 mM potassium chloride, 3 μM zinc sulfate, and 5 mM 2-mercaptoethanol. The frozen marine alga (Gracilaria chorda wet mass equal to 500 g) pulverized finely was homogenized by adding thereto 40 ml of the buffer solution for extraction, and this homogenized solution was kept standing at 4° C. for 6 hours and centrifuged to obtain a crude extract as the supernatant.

Subsequently, ammonium sulfate was added in a final concentration of 35% saturation to this crude extract to perform a first stage of salting-out. After the addition of the ammonium sulfate, the mixture was kept standing at 4° C. for 1 hour, and the generated precipitates were removed by centrifugation. This procedure eliminated impurities such as pigments as a precipitate fraction. Next, ammonium sulfate was added in a final concentration of 70% saturation to the supernatant obtained by the centrifugation. The mixture was kept standing overnight at 4° C., and the generated precipitates were separated by centrifugation. The separated precipitate fraction was redissolved in 100 mM phosphate buffer solution (pH 6.9) containing 0.15 M sodium chloride and dialyzed against 100 mM phosphate buffer solution (pH 6.9) containing 0.15 M sodium chloride to obtain a crude active fraction. The obtained crude active fraction had hemagglutination activity of 512 units against rabbit erythrocytes and specific activity of 6948 units/mg of proteins. In this context, the unit of hemagglutination activity was defined as the reciprocal of the maximum dilution rate of a sample from which the hemagglutination activity was detectable.

Gracilaria chorda (growing in the estuary of the Katsuura River) (wet mass: 25 g) obtained on the third year of culture was washed with 0.15 M sodium chloride aqueous solution and frozen at −30° C. A buffer solution for extraction used was a 0.5 M tris(hydroxymethyl)aminomethane hydrochloride buffer solution (pH 8.2) containing 30 mM potassium chloride, 3 μM zinc sulfate, and 5 mM 2-mercaptoethanol. The frozen marine alga (Gracilaria chorda wet mass equal to 500 g) pulverized finely was homogenized by adding thereto 40 ml of buffer solution for extraction, and this homogenized solution was kept standing at 4° C. for 6 hours and centrifuged to obtain a crude extract as the supernatant.

Subsequently, ammonium sulfate was added in a final concentration of 35% saturation to this crude extract to perform the first stage of salting-out. After the addition of the ammonium sulfate, the mixture was kept standing at 4° C. for 1 hour, and the generated precipitates were removed by centrifugation. This procedure eliminated impurities such as pigments as a precipitate fraction. Next, ammonium sulfate was added in a final concentration of 70% saturation to the supernatant obtained by the centrifugation. The mixture was kept standing overnight at 4° C., and the generated precipitates were separated by centrifugation. The separated precipitate fraction was redissolved in 100 mM phosphate buffer solution (pH 6.9) containing 0.15 M sodium chloride and dialyzed against 100 mM phosphate buffer solution (pH 6.9) containing 0.15 M sodium chloride to obtain a crude active fraction. The obtained crude active fraction had hemagglutination activity of 512 units against rabbit erythrocytes and specific activity of 6810 units/mg of proteins. The results are shown in Table 5.

TABLE 5

Crude active fraction

| | | Hemagglutination | |
|---|---|---|---|
| | | activity*⁾ | Specific activity |
| | | (unit) | (unit/mg protein) |
| Unialgal culture | on the fourth | 512 | 6948 |
| strain (Example 2) | week of | | |
| | culture | | |
| | on the third | 512 | 6810 |
| | year of | | |
| | culture | | |
| Unialgal culture | on the fourth | 256 | 3204 |
| strain (Comparative | week of | | |
| Example 1) | culture | | |
| Unialgal culture | on the fourth | 256 | 3063 |
| strain (Comparative | week of culture | | |
| Example 2) | | | |

*⁾The hemagglutination activity was obtained by successively diluting the crude active fraction and calculating from the maximum dilution rate indicating the hemagglutination activity.

The crude active fractions thus obtained were measured for mitogenic activity, and a blastoid transformation test of human lymphocytes was conducted.

Next, a blastoid transformation test of human lymphocytes was conducted by ³H-thymidine incorporation to measure mitogenic activity to the purified preparation of the crude active fraction. In this case, the preparation of all materials for cell culture, for example a microplate, cell harvester, glass fiber filter, counting vial, ³H-thymidine, toluene scintillator (0.1 g of POPO+5 g of PPO/liter of toluene), and liquid scintillation counter, and all procedures using them were aseptically performed.

Next, an aqueous solution in the proportions of 100 ml of pure water dissolving therein 1.05 g of medium (manufactured by Bio-Whittaker; product name “RPMI 1640”), 0.2 g of sodium hydrogencarbonate, 10000 units of penicillin, 10 mg of streptomycin, and 10 ml of fetal bovine serum was prepared as a culture solution and subjected to filter filtration and sterilization. The culture solution was fed into vials according to the amount of the usage and stored at −20° C. with the vial hermetically sealed. The culture solution could be stored and used in this state for 2 months. When the culture solution was used, the vial was opened, and the culture solution in the vial was used up. Freezing and thawing were not repeated.

Lymphocytes were separated from heparin-supplemented blood by the Ficoll-Conray method, then washed three times with CMF-PBS (pH 7.0), and suspended in 1 ml of culture solution to calculate the number of lymphocytes. Subsequently, the number of lymphocytes was adjusted to 5×10⁵ cells/ml with the culture solution.

The lymphocytes were cultured by dispensing the lymphocytes suspension at 200 μl/well to a microplate. Subsequently, the lymphocyte-containing microplate was kept standing in a clean booth for 30 minutes, and the crude active fraction and phosphate buffer solution (PES) were dispensed as a mitogen solution at 20 μl/well to the microplate. Solutions diluted (10-fold to 320-fold) with a buffer solution were prepared from the crude active fraction and subjected to the experiment. The incorporated amount (cpm) of ³H-thymidine in the crude active fraction was determined by multiplying a measurement value in the diluted solution by a dilution magnification and calculating a value in terms of the undiluted solution.

Subsequently, the lymphocytes were cultured for 3 days under humid conditions at 37° C. in the air containing 5% CO₂. Eight hours before the completion of the culture, ³H-thymidine was dispensed at a final concentration of 1 μCi/ml of culture solution/well.

Activity measurement was performed as follows: the cells in the wells were harvested in a saline solution by use of Labo-MASH or the like while being gathered onto a glass fiber filter and continuously aspirated to wash the cells on the filter (for approximately 20 sec; approximately 1.5 ml of physiological saline solution). Then, the cell adherence portion on the glass filter was peeled off, then placed into a counting vial, and well dried. A liquid scintillator was dispensed at 5 ml/vial with a dispenser to perform the measurement with a scintillation counter. Lymphocytes from three human samples (hereinafter, referred to as sample I, sample II, and sample III) were used in the evaluation of the crude active fraction obtained from the unialgal culture strain of the alga on the fourth week of culture to conduct the experiment. The number of experiments conducted under fixed experimental conditions was set to 3, and the average value of these three measurements was determined. The result thereof is shown in Table 6. Moreover, lymphocytes from three human samples (hereinafter, referred to as sample IV, sample V, and sample VI) were used in the evaluation of the crude active fraction obtained from the unialgal culture strain of the alga on the third year of culture to conduct the experiment. The number of experiments conducted under fixed experimental conditions was set to 3, and the average value of these three measurements was determined. The result thereof is shown in Table 7.

TABLE 6

| | Incorporated amount of ³H-thymidine, cpm | | |
|---|---|---|---|
| | Sample | Sample | Sample |
| | I | II | III |
| Crude active fraction | 91460 | 111460 | 90800 |
| obtained from the | | | |
| unialgal culture strain | | | |
| on the fourth week of | | | |
| culture (Example 2) | | | |
| Crude active fraction | 45970 | 53080 | 39480 |
| obtained from the | | | |
| unialgal culture strain | | | |
| on the fourth week of | | | |
| culture (Comparative | | | |
| Example 1) | | | |
| Crude active fraction | 30200 | 39080 | 26400 |
| obtained from the | | | |
| unialgal culture strain | | | |
| on the fourth week of | | | |
| culture (Comparative | | | |
| Example 2) | | | |
| Negative control (PBS) | 348 | 268 | 243 |

TABLE 7

| | Incorporated amount of ³H-thymidine, cpm | | |
|---|---|---|---|
| | Sample | Sample | Sample |
| | IV | V | VI |
| Crude active fraction | 86200 | 102100 | 89420 |
| obtained from the | | | |
| unialgal culture strain | | | |
| on the third year of | | | |
| culture (Example 2) | | | |
| Negative control (PBS) | 264 | 320 | 298 |

Since marine macroalgae have the ability to absorb nutritional salts such as nitrate nitrogen, phosphate ions, ammonium ions (nitrogen), the maximum amount of nitrate nitrogen absorbed per day was evaluated as the ability of the unialgal culture strain to absorb nutritional salts.

The maximum loading of nitrate ions per unit wet mass on the fourth week of culture of the unialgal culture strain prepared from the spores of the marine alga of the genus of Gracilaria sp. growing in the Katsuura River (Gracilaria chorda growing in the Katsuura River) was approximately 0.4 mg of nitrogen/g of marine alga wet mass/day. The result is shown in Table 8. The maximum daily loading of nitrate ions per unit wet mass on the third year of culture thereof was also approximately 0.4 mg of nitrogen/g of marine alga wet mass/day.

TABLE 8

| | | The maximum loading of nitrate |
|---|---|---|
| | | nitrogen absorbed per day |
| | | (mg of nitrogen/g of marine alga |
| | | wet mass/day) |
| Unialgal | on the fourth | 0.4 |
| culture strain | week of | |
| (Example 2) | culture | |
| | on the third | 0.4 |
| | year of | |
| | culture | |
| Unialgal | on the fourth | 0.2 |
| culture strain | week of | |
| (Comparative | culture | |
| Example 1) | | |
| Unialgal | on the fourth | 0.1 |
| culture strain | week of | |
| (Comparative | culture | |
| Example 2) | | |

### COMPARATIVE EXAMPLE 1

Unialgal culture strain was obtained in the same way as in Example 2 except that Gracilaria chorda (growing in the estuary of the Yoshino River) was used as a raw material instead of the marine alga of the genus of Gracilaria sp. growing in the Katsuura River (Gracilaria chorda growing in the Katsuura River).

As a result of maturation evaluation and growth speed measurement of the unialgal culture strain prepared from the Gracilaria chorda (growing in the estuary of the Yoshino River), maturation was observed in 12 weeks both in culture with 400 ml of culture solution and in culture with 20 liters of culture solution. The growth rate was 8.2%/day, and the mass after 4 weeks of culturing of 4 g of the marine alga was 12 g, which was lower than that of the immaturable unialgal culture strain prepared from the “marine alga of the genus of Gracilaria sp. growing in the Katsuura River (Gracilaria chorda growing in the Katsuura River)” (Table 4). Hemagglutination activity contained therein was 256 units for the crude active fraction and 3204 units/mg of proteins as specific activity, which were lower than those of the immaturable unialgal culture strain prepared from the “marine alga of the genus of Gracilaria sp. growing in the Katsuura River (Gracilaria chorda growing in the Katsuura River)” (Table 5). The mitogenic activity thereof against all the three human samples was lower than that of the immaturable unialgal culture strain prepared from the “marine alga of the genus of Gracilaria sp. growing in the Katsuura River (Gracilaria chorda growing in the Katsuura River)” (Table 6). The maximum daily acceptable loading of nitrogen was 0.2 mg of nitrogen/g of marine alga wet mass/day, which was one half the value of the immaturable unialgal culture strain prepared from the “marine alga of the genus of Gracilaria sp. growing in the Katsuura River (Gracilaria chorda growing in the Katsuura River)” (Table 7).

### COMPARATIVE EXAMPLE 2

Unialgal culture strain was obtained in the same way as in Example 2 except that Gracilaria chorda growing offshore of Komatsushima was used as a raw material instead of the marine alga of the genus of Gracilaria sp. growing in the Katsuura River (Gracilaria chorda growing in the Katsuura River).

As a result of maturation evaluation and growth speed measurement of the unialgal culture strain prepared from the Gracilaria chorda growing offshore of Komatsushima, maturation was observed in 11 weeks both in culture with 400 ml of culture solution and in culture with 20 liters of culture solution. The growth rate was 7.7%/day, and the mass after 4 weeks of culturing of 4 g of the marine alga was 11 g, which was lower than that of the immaturable unialgal culture strain prepared from the “marine alga of the genus of Gracilaria sp. growing in the Katsuura River (Gracilaria chorda growing in the Katsuura River)” (Table 4). Hemagglutination activity contained therein was 256 units for the crude active fraction and 3063 units/mg of proteins as specific activity, which were lower than those of the immaturable unialgal culture strain prepared from the “marine alga of the genus of Gracilaria sp. growing in the Katsuura River (Gracilaria chorda growing in the Katsuura River)” (Table 5). The mitogenic activity thereof against all the three human samples was lower than that of the immaturable unialgal culture strain prepared from the “marine alga of the genus of Gracilaria sp. growing in the Katsuura River (Gracilaria chorda growing in the Katsuura River)” (Table 6). The maximum daily acceptable loading of nitrogen was 0.1 mg of nitrogen/g of marine alga wet mass/day, which was one fourth the value of the immaturable unialgal culture strain prepared from the “marine alga of the genus of Gracilaria sp. growing in the Katsuura River (Gracilaria chorda growing in the Katsuura River)” (Table 7).

The respective results show that the unialgal culture strain prepared from the spores of the marine alga of the genus of of Gracilaria sp. growing in the Katsuura River (Gracilaria chorda growing in the Katsuura River) are immaturable even after 3 years or longer of continued culturing under normal culture conditions and are red algae belonging to the genus of Gracilaria sp. having at least one of the following three characteristic properties: properties of (1) producing a biologically active substance in a high yield, (2) showing a high growth speed of the alga body, and (3) being capable of readily absorbing nutritional salts.

The unialgal culture strain prepared from the spores of the marine alga of the genus of Gracilaria sp. growing in the Katsuura River (Gracilaria chorda growing in the Katsuura River) have advantages of (1) being immaturable, (2) being produced in a high yield, (3) having a high biologically active substance content, and (4) being capable of readily absorbing nutritional salts, as compared with the unialgal culture strain prepared from the spores of Gracilaria chorda (growing in the estuary of the Yoshino River) and with the unialgal culture strain prepared from the spores of Gracilaria chorda growing offshore of Komatsushima, and are advantageous in an industrial practice.

### EXAMPLE 3

Immaturable unialgal culture strain was prepared in the same way as in Example 2 from the spores of Gracilaria chorda growing in the estuary of the Katsuura River, and these culture strains were continuously cultured for 5 years. When the culture strain was measured for the number of other algae adhering to the surface thereof found by microscopy, it was smaller than 10 cells per 400 mg of culture strain wet mass.

For comparison, Gracilaria chorda growing offshore of Komatsushima was collected form a natural sea area and washed three times with the seawater medium described in Example 2 to measure other algae adhering to the surface thereof found by microscopy. As a result, the adherence of approximately 70000 cells per 400 mg of Gracilaria chorda wet mass was already observed. The naturally collected alga body of this Gracilaria chorda growing offshore of Komatsushima was further washed 10 times with the seawater medium described in Example 2, then chopped to a length of 3 cm, and further washed 10 times with the seawater medium described in Example 2 to obtain washed segments. When the culture of these washed segments in the seawater medium described in Example 2 was initiated, microalgae conspicuously grew in a flask containing the marine alga segments on the 14th day into the culture, reducing increase in the marine alga wet mass. As a result, the marine alga wet mass on the 21st day of the culture had fallen lower than that on the 14th day of the culture.

This shows that the immaturable unialgal culture strain of the present invention possesses the property of resisting the growth of adhering algae.

### INDUSTRIAL APPLICABILITY

An immaturable unialgal culture strain of the present invention is resistant against adherence of adhering algae and as such, has an advantage of causing no contamination with impurities and toxic components derived from the adhering algae in collecting useful substances from the alga body after growing. Additionally, the immaturable unialgal culture strain is derived from a marine macroalga of red algae having at least one of the following properties (1) to (3): properties of (1) producing a biologically active substance in a high yield, (2) showing a high growth speed of the alga body, and (3) being capable of readily absorbing nutritional salts. Therefore, it can be cultured or stored over a long period of time while being immaturable. The unialgal culture strain is preferably used, for example, in the production of biologically active substances such as hemagglutination agents.

1. An immaturable unialgal culture strain derived from a marine macroalga of red algae growing in a natural seawater area with intermixing of fresh water and having characteristics that no female gametophytes are detectable as matured bodies in nature and only tetrasporophytes are detectable as matured bodies.

2. The immaturable unialgal culture strain according to claim 1, wherein the marine macroalga of red algae is a red alga belonging to the genus of Gracilaria sp.

3. The immaturable unialgal culture strain according to claim 2, wherein the red alga belonging to the genus of Gracilaria sp. is Gracilaria verrucosa, Gracilaria chorda, or a subspecies thereof.

4. The immaturable unialgal culture strain according to claim 1, wherein the amount of adhering algae is smaller than 10 cells per 400 mg of the wet mass amount after 3 years of continued culturing.

5. A method for producing an immaturable unialgal culture strain characterized by the steps of collecting matured sporophytes of a marine macroalga of red algae growing in a natural seawater area with intermixing of fresh water and having characteristics that no female gametophytes are detectable as matured bodies in nature and only tetrasporophytes are detectable as matured bodies, standing the sporophytes as cut open to cause release of the spores and culturing the released spores to continue growing and culturing after sprouting of upright bodies from germinated spores.

6. The method for producing an immaturable unialgal culture strain according to claim 5, wherein the marine macroalga of red algae is a red alga belonging to the genus of Gracilaria sp.

7. The method for producing an immaturable unialgal culture strain according to claim 6, wherein the red alga belonging to the genus of Gracilaria sp. is Gracilaria verrucosa, Gracilaria chorda , or a subspecies thereof.

8. The method for producing an immaturable unialgal culture strain according to claim 5, wherein the natural seawater area with intermixing of fresh water is a seawater area where the salt content does not exceed 1.0% by mass.

9. The method for producing an immaturable unialgal culture strain according to claim 5, wherein the immaturable unialgal culture strain has adhering algae of smaller than 10 cells in number per 400 mg of wet mass amount after 3 years of continued culturing.

10. An alga body obtained by growing the immaturable unialgal culture strain according to claim 1.

11. The alga body according to claim 10, wherein the marine macroalga of red algae is a red alga belonging to the genus of Gracilaria sp.

12. The alga body according to claim 11, wherein the red alga belonging to the genus of Gracilaria sp. is Gracilaria verrucosa, Gracilaria chorda, or a subspecies thereof.

13. The alga body according to claim 12, wherein the amount of adhering algae is smaller than 10 cells per 400 mg of the wet mass amount after 3 years of continued culturing.
